(19) United States
(12) Patent Application Publication
(10) Pub. No.: US 2023/0241080 A1
(43) Pub. Date: Aug. 3, 2023

(54) TRIAMCINOLONE ACETONIDE-LOADED LIPOSOMES TOPICAL OPHTHALMIC FORMULATIONS AS PRIMARY THERAPY FOR MACULAR EDEMA SECONDARY TO BRANCH RETINAL VEIN OCCLUSION

(71) Applicants: Arturo SANTOS, Zapopan, Jalisco (MX); OPKO Pharmaceuticals, LLC, Miami, FL (US)

(72) Inventors: Arturo SANTOS, Zapopan (MX); Jose NAVARRO, Guadalajara (MX); Juan C. ALTAMIRANO, Guadalajara (MX); Alejandro GONZALEZ, Guadalajara (MX)

(21) Appl. No.: 18/010,099

(22) PCT Filed: May 4, 2021
(86) PCT No.: PCT/US2021/030593

§ 371 Date: Dec. 13, 2022

Related U.S. Application Data
(60) Provisional application No. 63/039,095, filed on Jun. 15, 2020.

(51) Int. Cl. A61K 31/58; A61K 47/14; A61K 9/00; A61P 27/02

(52) U.S. Cl. CPC A61K 31/58; A61K 47/14; A61K 9/0048; A61P 27/02

(57) ABSTRACT

The recited invention is a method of treating patients having macular edema secondary to branch renal vein occlusion. The patients are treated with a liposomal nanoparticle formulation comprising thermodynamically stable liposomes and a steroid such as triamcinolone acetonide. The formulation is a topical ophthalmic formulation administered topically to treat the posterior segment disease.

### CROSS-REFERENCE TO RELATED APPLICATION

The application claims benefit of U.S. Provisional Patent Application Ser. No. 63/039,095, filed on Jun. 15, 2020, which is incorporated herein by reference in their entirety.

### BACKGROUND OF THE INVENTION

Retinal vein occlusion is the second most common cause of retinal vascular disease after diabetic retinopathy¹. Population studies suggest that approximately 16 million people in the world suffer from central or branch retinal vein occlusion (CRVO and BRVO respectively). BRVO represents the typical presentation with a prevalence of 4.42 per 1000, while CRVO has just a prevalence of 0.80 per 1000². The clinical relevance of BRVO is that it usually impairs visual function permanently³. BRVO can be divided into two different types; major BRVO, when one of the major branch retinal vein is occluded, and macular BRVO, when one of the macular venules is occluded^{4 5}. Major BRVO comprises a nonischemic form and an ischemic form that could progress to neovascularization⁴.

Different risk factors have been related to BRVO such as; hypertension (HTN), hyperlipidemia (HLD), peripheral arterial disease (PAD) and diabetes mellitus (DM)⁶⁻⁸. Moreover, other less common ocular and non-ocular conditions have been associated to BRVO, for example; inadvertent retrobulbar needle perforation, axial length, vitreous chamber depth, posterior vitreous adhesion, liver or renal diseases, and obstructive sleep apnea (OSA)⁹⁻¹⁴.

The leading cause of impaired vision in patients with BRVO is macular edema (ME), and occurs in approximately 30% of BRVO cases¹⁵. Presumably this phenomenon appears as result of the efflux of fluid from the affected vessels to the retinal tissue, due to breakdown of the blood-retinal barrier, damage to the tight junctions between capillary endothelial cells, vitreoretinal adhesion and traction on the macula, and secretion into the vitreous of vasopermeability factors such as vascular endothelial growth factor (VEGF)¹⁶⁻¹⁸. It is well known that vascular occlusion induces the expression of (VEGF) in patients with BRVO¹⁹⁻²⁰. However, aqueous levels of other growth factors, cytokines and soluble receptors have been significantly correlated with CME such as interleukins (IL) 6, 8, 12 and 13, placental growth factor (PIGF), platelet-derived growth factor (PDGF)-AA, soluble intercellular adhesion molecule-1 (sICAM-1), monocyte chemoattractant protein-1 (MCP-1), aqueous angiopoietin-like 4 (ANGPTL4), soluble vascular endothelial growth factor receptor-1 (sVEGFR)-1 and sVEGFR-2²⁰⁻²⁴.

Treatment options for CME secondary to BRVO include macular grid laser photocoagulation and intravitreal (IVT) injections of steroids or anti-VEGF molecules^{25,26}. Laser therapy improves oxygenation to the treated area causing constriction of the occluded vein and the adjacent arteriole resulting decreased edema¹⁶, while anti-VEGF drugs block the signaling of the principal vasopermeability factor. Efficacy of laser treatment is limited compared with antiVEGF therapy and corticosteroids^{27,28}. On the other hand, steroids such as triamcinolone acetonide (TA) and dexamethasone acetate have antiinflammatory and antiangiogenic properties that inhibit the expression of VEGE and other proinflammatory cytokines²⁹. Because multiple cytokines are connected to the pathogenesis of the CME secondary to BRVO, the broad therapeutic spectrum of steroids like TA is desirable. However, the use of intravitreal TA is associated to severe adverse events such as endophthalmitis, lens injury, and retinal detachment³⁰⁻³². Additionally, clinical studies have related the use of intravitreal TA with intraocular pressure (IOP) increase, cataract formation or progression and noninfectious endophthalmitis³³⁻³⁵.

To diminish ocular hazards related to intravitreal injections of steroids, it is necessary to develop alternative strategies for drug delivery into the posterior segment of the eyeball (vitreous and retina). Nanostructured carriers or nanocarries (nanomaterials) have arisen as effective and slightly invasive drug delivery systems, which can keep drug concentrations in the posterior segment of the eyeball preventing the use of IVT injection or reducing its frequency. The advantageous to use nanocarriers are related to their capacity to increase the biopharmaceutical properties of the incorporated drug, including solubility, stability, permeability, and retention at the site of application³⁶.

Nanocarriers are composed of nanoparticles (NPs) (1-1000 nm) and constitutes one of the multiple strategies of the nanomedicine, interpreted as the application of NPs for medical purposes³⁷. Liposomes (LPs) are particles composed of an aqueous core and delimited by a membrane-like lipid bilayer that works as carriers for water-soluble, lipid-soluble and amphiphilic drugs³⁸⁻⁴¹. LPs are non-toxic, low antigenic, easily metabolized and biodegradable⁴² and they have been employed to improve drug transport and bioavailability in ocular tissues^{43,44}.

Liposomes-based eye drops have been proposed as a drug delivery system into the posterior segment of the eye, and they have the potential to deliver drugs like TA in therapeutic concentrations to the vitreous cavity and retina⁴⁵. Recently, a topical triamcinolone acetonide-loaded liposomes formulation (TA-LF) was used to successfully deliver triamcinolone acetonide (TA) into vitreous and retina of rabbits⁴⁵ and its therapeutic activity was confirmed in patients with refractory pseudophakic cystoid macular edema⁴⁶. In order to take advantage of the biological activity of steroids for the treatment of ME secondary to BRVO, but avoiding the risks of IVT route, Triamcinolone acetonide-loaded liposomal topical ophthalmic formulations (TA-LFs) ARE used as primary therapy in patients with ME secondary to BRVO. Applicant has filed multiple patent applications and which include a continuation-in-part application having U.S. Application No. [ ] filed on May [ ], 2019 which is a CIP of U.S. application Ser. No. 14/422,587 filed on Feb. 19, 2015 which is a national phase application of PCT/US2013/055084 filed on Aug. 15, 2013, all of which are incorporated by reference.

### SUMMARY OF THE INVENTION

The compositions of the present invention (a formulation) comprise a combination of triamcinolone acetonide as the active pharmaceutical ingredient, polyethyleneglycol (PEG-12) glyceryl dimyristate as structural constituent of liposomes, ethyl alcohol as organic solvent for liposomes generation, kolliphor HS 15 as penetration enhancer, citric acid anhydrous and sodium citrate dehydrate as buffers, benzalkonium chloride as preservative, and grade 2 purified water as solvent.

The formulations of the present invention are useful as primary therapy in the prevention or limitation of macular thickening and or macular cysts occurrence after a branch retinal vein occlusion event, and its associated visual outcomes, such as; visual acuity and contrast sensitivity.

### BRIEF DESCRIPTION OF THE DRAWINGS

FIG. 1 shows a SEM analysis indicating that that liposome size depends upon the concentration of triamcinolone acetonide (“TA”) in the liposome formulation. As the concentration of TA increased, the average size of liposomes and the number of particles with a diameter>1000 nm, increased as well.

FIG. 2 shows a TEM study displaying that the liposome formulation is able to solubilize large TA crystals into nanoparticles and encapsulate them at the same time.

FIG. 3 shows optical coherence tomography (OCT) images of all 12 patients.

FIG. 4 shows the differences in CFT, BCVA and IOP during follow-up of TA-LF therapy in the study and fellow eyes (eyes receiving TA-LF and eyes without topical therapy, respectively) of patients with ME secondary to BRVO.

### DETAILED DESCRIPTION OF THE INVENTION

Ingredient concentrations are presented in units of % weight/volume (% w/v) or % volume/volume (% v/v).

The compositions of the present invention contain a pharmaceutically effective amount of triamcinolone acetonide (TA). The concentration of TA in liposomes formulations ranges from 0.01 to 2.00% (w/v). TA is a known synthetic corticosteroid with an empirical formula of C₂₄H₃₁FO₆ and a molecular weight of 434.50 Da. TA has a powerful anti-inflammatory activity (7.5 times more potent than cortisone)⁴⁷. Polyethyleneglycol (PEG-12) glyceryl dimyristate is used as structural constituent of liposomes in a concentration of 5-15% (w/v) and ethyl alcohol is used as organic solvent for liposomes generation in a concentration of 0.7 to 2.1% (v/v).

The topical liposomal formulation further comprises polyethylene glycol (15)-hydroxystearate or Kolliphor HS 15 from 2.5-7.5% (w/v), as a potent non-ionic solubilizer and emulsifying agent, with low toxicity proposed to act as a permeability enhancer. Kolliphor HS 15 promotes drug transport across cell membranes (increasing the endocytosis rate) and stimulates drug translocation through the paracellular route (affects actin organization on the cell cytoskeleton with the subsequent tight junction opening)⁴⁸.

Additionally, the aqueous compositions of the present invention optionally comprise more excipients selected from the group consisting of buffering agents, pH-adjusting agents, and preservatives. Citric acid anhydrous (0.04-0.16%) and sodium citrate dehydrate (0.23-0.69%) are used as buffers, whereas benzalkonium chloride (0.001-0.015%) as preservative, all described in units of % w/v.

The compositions of the present invention may be prepared by conventional methods of preparing pharmaceutical suspension compositions. According to the preferred method, the drug (triamcinolone acetonide) is first added to a lipid mixture containing polyethyleneglycol (PEG-12) glyceryl dimyristate and ethyl alcohol. An aqueous mixture having grade 2 purified water, polyethylene glycol (15)-hydroxystearate (Kolliphor HS 15), citric acid anhydrous, sodium citrate dihydrate and benzalkonium chloride is comingled in a flask and set aside for compounding. The water mixture is gently added to the lipid mixture to obtain the final formulation.

The following example are intended to illustrate, but not limit, the present invention.

### Example 1

The formulations shown below is representative of the compositions of the present invention and is called TA-LF.

TABLE 1
Triamcinolone acetonide-loaded liposomes topical
ophthalmic formulation (TA-LF)

| | (w or v) | | (%) | |
|---|---|---|---|---|
| Triatncinolone aceletnide | 2.0 | mg | 0.2 | w/v |
| Kolliphor HS 15 | 50 | mg | 5 | w/v |
| PEG-12 glyceryl dimyristate | 100 | mg | 10 | w/v |
| Ethyl alcohol | 14 | mL | 1.40% | v/v |
| Citric acid anhydrous | 0.8 | mg | 0.0s | w/v |
| Sodium citrate dihydrate | 4.675 | mg | 0.4675 | w/v |
| Benzalkonium chloride | 0.1 | mg | 0.01 | w/v |
| Grade 2 purified water | Q.S. 1.0 | ml | NA | NA |

NA; not applicable, v; volume, w, weight

The formulations shown in Table 1 were prepared and subjected to a physicochemical characterization. pH of TA-FL was analyzed by a pH meter in triplicate at room temperature. Osmolarity was measured by a vapor pressure osmometer and performed in triplicate at 33° C. (the ocular surface temperature)⁴⁹. Viscosity was measured also in triplicate at 33° C. Viscosity was measured using a thermostatically controlled rheometer when the steady state was reached with shear rates increasing from 0 to 1000 s-1. Particle size of the TA-LFs was analyzed by means of Dynamic Light Scattering and zeta potential (ζ) was calculated by measuring the velocity of the particles using Laser Doppler Velocimetry at 25° C. (Zetasizer Nano ZS, Malvern Instruments, Malvern, UK). The Z-average (mean particle diameter) and polydispersity index (PDI) were calculated from the particle size distribution.

TABLE 2
Physicochemical properties of TA-LF.

| | Physicochemical parameters | | | | |
|---|---|---|---|---|---|
| | | | | Z- | |
| | | Viscosity | Osmolarity | averge | PDI |
| Formulation | pH | (cP) | (mOsm/l) | (nm) | (nm) |
| TA-LF | 5.8 | 70 | 334 | 187.8 | 0.369 |

pH = hidrogenion potential, cP = centipoise, PDI = polydispersity index Values represent the average of three measures

Posteriorly, TA-LF from example I was evaluated in an in vitro diffusion assay. Diffusion chambers and rabbit corneas were used to conduct diffusion experiments (Chemotaxis Chambers BW200S, NeuroProbe, Gaithersburg, Md., USA). Rabbit corneas from New Zealand white rabbits were used for this experiment. The central corneal tissue was located between the top and bottom compartments of the diffusion chambers to act as a TA diffusion barrier. The top compartment was filled with 180 μl of balanced salt solution (BSS) while the bottom compartment was filled with 200 μl of TA-LFs (TA-LF1 to TA-LF4). To avoid evaporation, the diffusion chambers were located into a 37° C. humidity camera. The TA concentration analysis of solutions obtained from the top compartment at 2, 4, 6 and 8 hours (h) after starting the diffusion assay, was performed by high performance liquid chromatography (HPLC). HPLC was performed using a Varian 920 LC (Aligent Technologies, Santa Clara, Calif., USA) with a Zorbax Eclipse Plus C18, 4.6×100 mm and 3.5-μm column (Agilent, Santa Clara, Calif., USA) at 30° C. The samples (20 μl) were eluted from the column in a mobile phase comprised of water:methanol (30:70) at a flow rate of 1 ml/min. Detection was performed at 254 nm. Retention time and detection limit were 6.8 min and 0.004 mg/ml respectively. The TA standard curve was linear from 0.004 to 0.100 mg/ml (correlative>0.99). In vitreous, concentrations of TA were determined for the recovery and intra- and inter-day reproducibility⁵⁰.

Result from the in vitro diffusion assay is provided in table 3.

TABLE 3

| TA concentration in the top | |
|---|---|
| solution of the diffusion chamber across | |
| time | |
| Time | |
| (hours) | TA Concentration (μg/mL) |
| 2 | 0.22 |
| 3 | 0.35 |
| 4 | 0.5 |
| 5 | 0.65 |
| 6 | 0.8 |
| 7 | 0.95 |
| 8 | 1.1 |

Values represent the average of three measures.

We observed that TA-LF presented the best diffusion performance, reaching the highest TA concentrations after 8 hours of follow up.

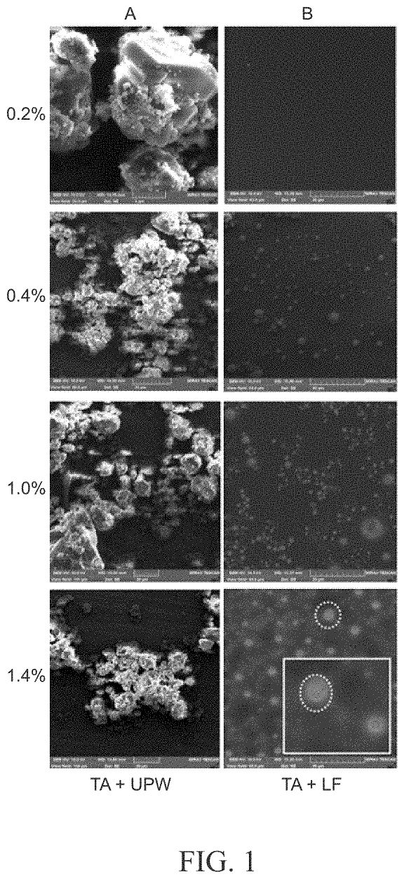

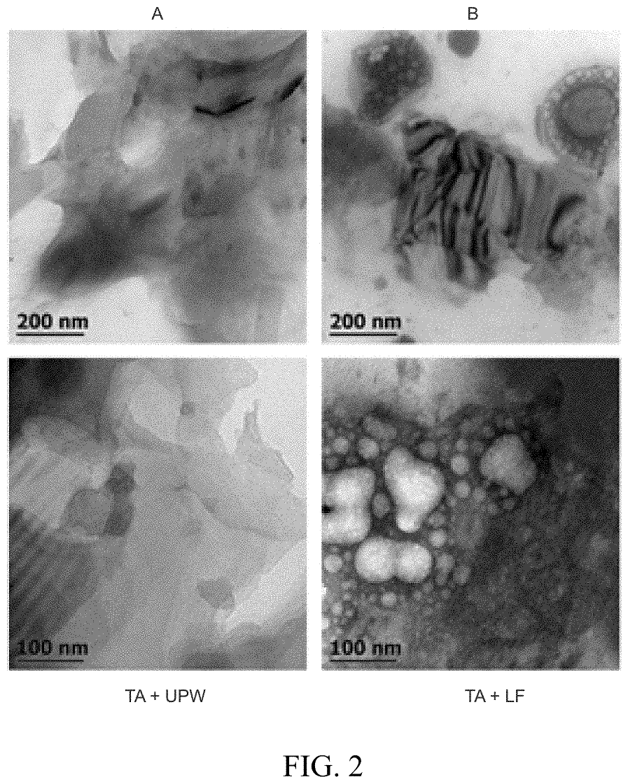

Posteriorly, morphology of TA crystal in aqueous solution and morphology of TA-LFs were assessed through Scanning Electron Microscopy (SEM) and also Transmission Electron Microscopy (TEM). Aqueous solutions of TA were prepared adding ultrapure water (UPW) to the required quantity of TA crystals (TA+UPW) to achieve the concentrations of 0.2, 0.4, 1.0 and 1.4%. Emulsions of TA loaded liposomes (TA+LF) were produced varying the quantity of TA crystals in the formulation described in table 1 to reach the concentration of 0.2, 0.4, 1.0 and 1.4% of the active ingredient. For SEM, a TESCAN MIRA3 LMU FE-SEM device was used, while for TEM a JEOL JEM-1010 electron microscope. SEM samples were kept at −4° C. before being mounted onto stubs and were gold-coated using a Denton Vacuum Desk II sputter coater. TEM samples were previously treated using phosphotungstic acid as negative staining agent in a 1:1 dilution (v/v) and were deposited onto FF 300 square mesh copper grids for observation. Manual count and measure of particles where performed in SEM micrographs at a view field of 63.6 □m to calculate the size and distribution of liposomes. SEM analysis showed that liposomes size depends on the concentration of TA in the liposome formulation. As the concentration of TA increased, the average size of liposomes and the number of particles with a diameter>1000 nm, increased as well (FIG. 1 and Table 4). For example, TA+LF 0.2% (the formulation used in this pilot study) presented an average particle size of 147.38 with 0% of particles superior than 1000 nm, whereas TA+LF 1.4% shown an average particle size of 1442.96 nm with 53% of particles greater than 1000 nm (results are presented in Table 4). On the other hand, TEM study displayed that the liposome formulation is able to solubilize large TA crystals into nanoparticles and encapsulate them at the same time (FIG. 2).

TABLE 4
Number of particles and particle size obtained from scanning electron
microscopy micrographs of triamcinelone acefonide-loaded liposomes formulations.

| | Triamcinolone acetonide loaded-liposomes formulation | | | |
|---|---|---|---|---|
| | (TA + LF) | | | |
| | 0.2% | 0.4% | 1.0% | 1.4% |
| Average size (nm) | 147 ± 6 | 855 ± 37 | 1159 ± 52 | 1443 ± 71 |
| Minimum particle | 111 | 259 | 259 | 333 |
| size (nm) | | | | |
| Maximum particle | 593 | 3222 | 3333 | 3666 |
| size (nm) | | | | |
| Relative frequency of particles (%) | | | | |
| <200 nm | 92.78 | 0 | 9 | 0 |
| 201-500 nm | 6.19 | 39.6 | 25 | 8 |
| 501-1000 nm | 1.03 | 26.73 | 30 | 39 |
| >1000 nm | 0 | 33.67 | 45 | >3 |

After in vitro assays and microscopic characterization, in vivo diffusion analysis and tolerability assessment of TA-LF was performed in rabbits. For diffusion analysis, concentrations of TA were determined by HPLC in ocular tissues from New Zealand white rabbits after multiple doses of TA-LF2. For tolerability assessment, eye examination of study animals was performed after topical administration of TA-LF. The protocol for animals was the following. Rabbits were randomly distributed into four groups. One-drop TA-LF2 solution (50 μl) was applied to one eye every two hours 6 times during 14 days. Five rabbits were sacrificed after starting the instillation of TA-LF2 at 12 hours, 1, 7 and 14 days. Before tissue collecting, an eye examination was performed under anesthesia (intramuscular injection of ketamine hydrochloride 30 mg/kg and chlorpromazine hydrochloride 15 mg/kg). This evaluation included slit-lamp biomicroscopy, fluorescein staining, funduscopy with direct ophthalmoscope, and intraocular pressure (IOP) measurement (iCare Tonometer i350, Vantaa, Finland). Additionally, ocular irritability test was evaluated according to pharmacopeia of Estados Unidos Mexicanos. A positive irritant reaction is considered when more than one rabbit presented: cornel ulceration revealed by fluorescein staining, corneal opacity, iris or conjunctival inflammation and dilatation of conjunctival vessels especially around the cornea. After enucleation, conjunctiva, cornea, retina, 150 μl of aqueous humor and 200 μl of vitreous were collected. The solid tissues were washed in PBS. Then, tissues were homogenized with 0.3 ml of acetonitrile (Sigma-Aldrich, Mexico). Posteriorly, each sample was centrifuged at 15,294×g for 5 min. The supernatants were evaporated to add 100 μl of methanol. Another centrifugation was performed and 20 μl of the resultant supernatants were used for analysis of TA concentration by HPLC, performed as previously described.

The concentrations of TA in retina and vitreous reached the highest peak at 12 hours (252.1±90.00 ng/g and 32.6±10.27 ng/g respectively) to subsequently decline to 24.0±11.72 ng/g and 19.5±13.14 ng/g respectively at 14 days of follow up. TA concentration vs time in different ocular tissues are presented in FIG. 1 and Table 4.

TABLE 4
Ocular tissues concentration of TA after topical administration of TA-LF in rabbit eyes.
Triamcinolone acetonide concentration (ng/g)

| Time | | | | | | |
|---|---|---|---|---|---|---|
| (days) | Conjunctiva | Cornea | Lens | Retina | Aqueous | Vitreous |
| 0.5 | 1886.3 □ 398.95 | 2156.1 □ 1055.41 | 83.3 □ 30.49 | 252.1 □ 90.00 | 9.9 □ 1.95 | 32.6 □ 10.27 |
| 1 | 1524.8 □ 356.04 | 657.5 □ 260.37 | 62.2 □ 18.54 | 196.9 □ 133.10 | 9.8 □ 2.12 | 18.6 □ 7.07 |
| 7 | 359.3 □ 166.54 | 132.5 □ 78.37 | 52.3 □ 37.42 | 57.7 □ 20.92 | 8.1 □ 2.71 | 20.7 □ 11.35 |
| 14 | 52.3 □ 27.82 | 92.3 □ 42.13 | 5.2 □ 3.38 | 24 □ 11.72 | 2.8 □ 0.13 | 19.5 □ 13.14 |

Values represent the average □□□ standard deviation of the mean of four samples

Compartmental and non-compartmental model were used to determine pharmacokinetics of TA-loaded liposomes in ocular tissues. Linear-trapezoidal method was employed to evaluate the area under the curve (AUC). The half-life (t_{1/2}) was calculated by linear regression of the concentration at different times. Pharmacokinetic parameters are shown in table 5.

Cₘₐₓ was 2156.07±1055.41 ng/g in cornea, 1886.33±398.95 ng/g in conjunctiva, 9.9±1.95 ng/g in aqueous humor, 83.3±30.49 ng/g in lens, 32.6±10.27 ng/g in vitreous and 252.10±90.00 ng/g in retina.

TABLE 5
Pharmacokinetics parameters in ocular tissues after topical administration of TA-FL.

| Parameter | Conjunctiva | Cornea | Aqueous | Lens | Vitreous | Retina |
|---|---|---|---|---|---|---|
| kₑ (d⁻¹) | 0.263 | 0.26 | — | | 0.79 | 0.26 |
| T_{1/2} (d) | 2.64 | 2.66 | 0.23 | 0.09 | 0.87 | 3.62 |
| Cₘₐₓ (ng/g) | 1886.33 | 2156.075 | 9.94 | 83.328 | 32.6 | 252.1 |
| Cₘᵢₙ (ng/g) | 52.26 | 92.348 | 2.795 | 5.183 | 18.613 | 24 |
| k₁₂ (d⁻¹) | — | 2.365 | — | — | 13.48 | 0.383 |
| k₂₁ (d⁻¹) | — | 0.045 | — | — | 0.018 | 0.478 |
| ABC0^{-t} (ng*g/d) | 7,945.35 | 3,860.50 | 96.75 | 610.052 | 271.378 | 1,159.40 |
| ABC0^{-∞} (ng*g/d) | 8,144.32 | 3,933.12 | — | — | 296.054 | 1,251.69 |

kₑ = elimination rate constant; d = day; T_{1/2} = elimination half-life Cₘₐₓ = observed maximum concentration; Cₘᵢₙ = observed minimum concentration; k₁₂ = rate of transfer central to peripheral compartment; k21 = rate of transfer from peripheral to central compartment; AUC^{0-t} = area under the curve until the last measurable; AUC^{0-∞} = area under the curve from 0 to infinity.

Related to tolerability assessment; no increase in intraocular pressure was observed in any of the study subjects (normal intraocular pressure in this species is 12-28 mmHg). Staining with fluorescein sodium and bengal rose showed superficial punctate keratitis in the first 6 hours after instillation of the formulation. This condition was resolved in all cases in the examination at 12 hours after the administration of the formulation. Therefore, according to pharmacopeia of Estados Unidos Mexicanos, ocular irritability test was satisfactory, and TA-LF2 is considered nonirritant.

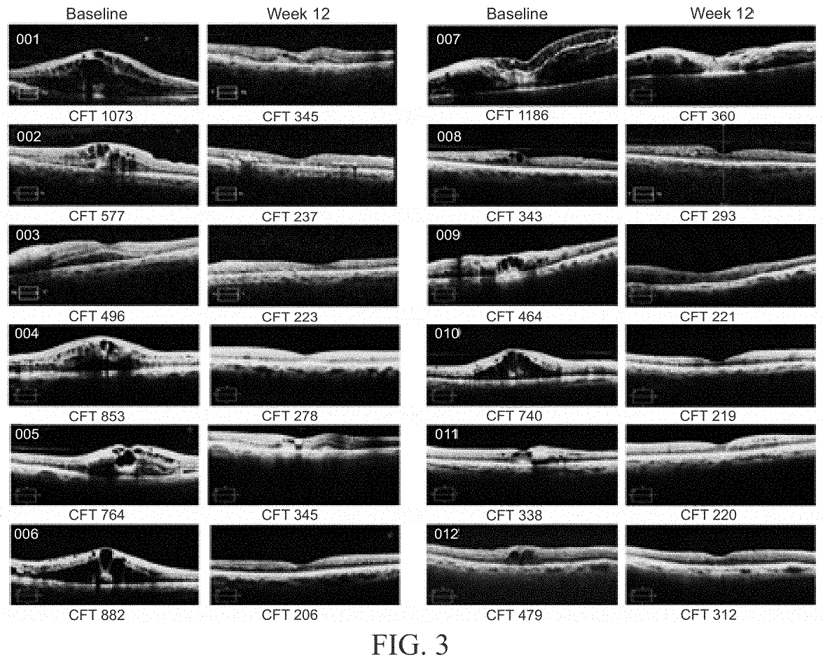

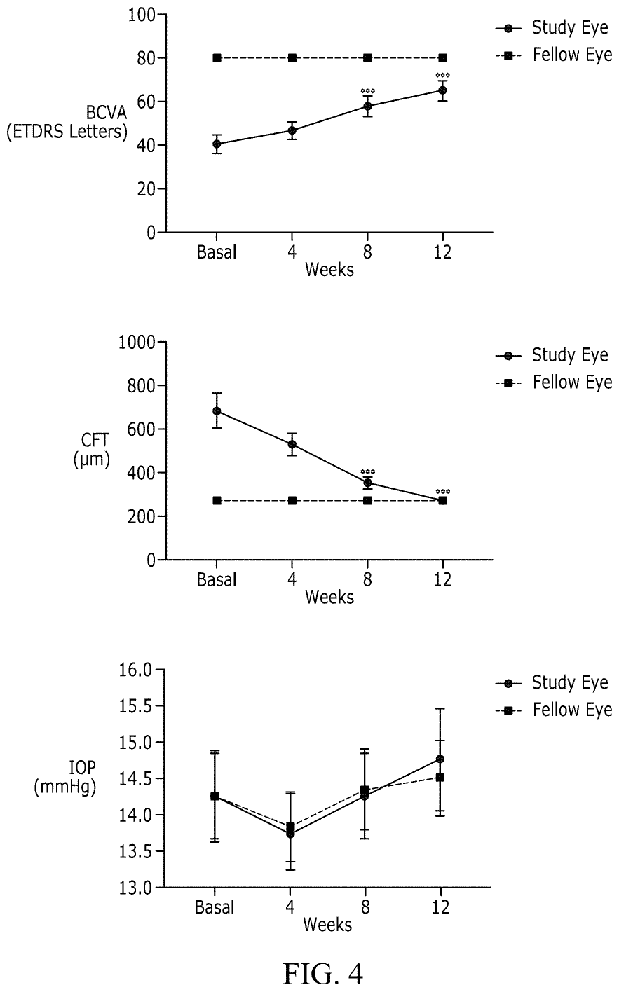

Finally, therapeutic activity of TA-LF as primary therapy for macular edema secondary to BRVO was proved in humans. For safety and efficacy evaluation, 12 eyes of 12 patients with ME secondary to BRVO were exposed to a topical instillation of one drop of TA-LF (TA 0.2%) six times daily for 12 weeks (demographics and clinical characteristics of patients are presented in table 6). Best corrected visual acuity (BCVA) Intraocular pressure (IOP), slit lamp examination and central foveal thickness (CFT) were analyzed at every visit. Patients with ME secondary to BRVO under TA-LF therapy presented a significant improvement of BVCA and CFT without significant IOP modification (P=0.94). Optical coherence tomography (OCT) images of all 12 patients are showed in FIG. 3. OCT is a noninvasive imaging technology used to obtain high-resolution cross-sectional images of the retina. The treated eyes also showed BCVA improvement from 40±12.05 to 64.83±15.97 letters, and CFT reduction from 682.91±278.60 to 271.58±57.66 □m after 12 weeks of TA-LF therapy (P<0.001). No adverse events including IOP rising were registered (variations in BCVA, CFT an IOP of patients with ME secondary to BRVO throughout TA-LF therapy are presented in table 7). Remarkably, non-significant variations in IOP were recorded between the study and the fellow eyes, supporting the safety of TA-LF (FIG. 4). Differences in CFT, BCVA and IOP during follow-up of TA-LF therapy in the study and fellow eyes (eyes receiving TA-LF and eyes without topical therapy, respectively) of patients with ME secondary to BRVO are indicated in FIG. 4. In conclusion, TA-LF can function as nanocarriers of TA and they could be used as topical ophthalmic primary therapy instead of intravitreal drugs in patients with ME secondary to BRVO.

TABLE 6
Demographics and Clinical Characteristics of patients
with ME secondary to BRVO treated with TA-LF.

| | | | Retinal Vein | | | | | |
|---|---|---|---|---|---|---|---|---|
| | | | Occlusion | | Comorbidities | | | |
| PAT | GENDER | AGE | EYE | VEIN | HTN | DM | OTHER | LENS STATUS |
| 1 | M | 80 | OD | TS | Yes | No | Ischemic | Pseudophakic |
| | | | | | | | disease | |
| 1 | F | 64 | OD | TS | Yes | No | No | Mild cataract |
| 3 | F | 59 | OS | TI | Yes | No | No | Phakic |
| 4 | M | 58 | OD | TS | Yes | No | No | Pseudophakic |
| 5 | M | 62 | OS | TS | Yes | No | No | Mild cataract |
| 6 | F | 86 | OS | TI | Yes | No | No | Pseudophakic |
| 7 | F | 78 | OS | TS | Yes | No | No | Pseudophakic |
| 8 | M | 70 | OD | TS | Yes | Yes | No | Pseudophakic |
| 9 | M | 74 | OD | TI | Yes | No | Ischemic | Pseudophakic |
| | | | | | | | disease | |
| 10 | M | 71 | OS | TS | Yes | No | No | Pseudophakic |
| 11 | M | 68 | OD | TS | Yes | No | No | Pseudophakic |
| 12 | F | 63 | OD | TS | Yes | No | No | Pseudophakic |

ME; macular edema, BRVO; branch retinal vein occlusion, DM; Diabetes mellitus, F; female, HTN; Hypertension, M; male, OD; right eye, OS; left eye, TA-LF; Triamcinolone acetonide loaded liposomes formulation, TS; temporal superior, TI; temporal inferior.

TABLE 7
Variations in BCVA, CFT and IOP of patients with
ME secondary to BRVO throughout TA-LF therapy.

| | BASELINE | | | WEEK 4 | | |
|---|---|---|---|---|---|---|
| | BCVA | | | BCVA | | |
| | (ETDRS | CFT | IOP | (ETDRS | CFT | IOP |
| PATIENT | letters) | (□m) | (mmHg) | letters) | (□m) | (mmHg) |
| 1 | 20 | 1073 | 15 | 27 | 835 | 13 |
| 2 | 29 | 577 | 17 | 33 | 485 | 12 |
| 3 | 48 | 496 | 13 | 58 | 312 | 14 |
| 4 | 45 | 853 | 15 | 49 | 583 | 12 |
| 5 | 47 | 764 | 10 | 50 | 623 | 13 |
| 6 | 44 | 882 | 18 | 49 | 692 | 15 |
| 7 | 21 | 1186 | 15 | 22 | 728 | 18 |
| 8 | 62 | 343 | 13 | 68 | 301 | 14 |
| 9 | 38 | 464 | 15 | 44 | 414 | 15 |
| 10 | 36 | 740 | 15 | 47 | 628 | 14 |
| 11 | 47 | 338 | 12 | 61 | 287 | 11 |
| 12 | 43 | 479 | 13 | 49 | 428 | 14 |
| | 40 ± | 682.91 ± | 14.25 ± | 46.41 ± | 526.33 ± | 13.75 ± |
| | 12.05 | 278.60 | 2.17 | 13.50‡ | 181.97‡ | 1.81‡ |

| | WEEK 8 | | | WEEk 12 | | |
|---|---|---|---|---|---|---|
| | BCVA | | | BCVA | | |
| | (ETDRS | CFT | IOP | (ETDRS | CFT | IOP |
| PATIENT | letters) | (□m) | (mmHg) | letters) | (□m) | (mmHg) |
| 1 | 42 | 462 | 12 | 58 | 345 | 16 |
| 2 | 38 | 315 | 14 | 45 | 237 | 13 |
| 3 | 72 | 248 | 14 | 74 | 223 | 16 |
| 4 | 72 | 302 | 17 | 78 | 278 | 17 |
| 5 | 57 | 461 | 12 | 62 | 345 | 14 |
| 6 | 59 | 438 | 16 | 79 | 206 | 16 |
| 7 | 22 | 501 | 13 | 30 | 360 | 14 |
| 8 | 76 | 290 | 12 | 80 | 293 | 10 |
| 9 | 53 | 263 | 17 | 52 | 221 | 18 |
| 10 | 69 | 322 | 12 | 80 | 219 | 11 |
| 11 | 69 | 232 | 16 | 68 | 220 | 17 |
| 12 | 62 | 370 | 16 | 72 | 312 | 15 |
| | 57.58 ± | 350.33 ± | 14.25 ± | 64.83 ± | 271.58 ± | 14.75 ± |
| | 16.37† | 93.23† | 2.05‡ | 15.97† | 57.66† | 2.45‡ |

ME; macular edema,
CFT; central foveal thickness,
BCVA; best corrected visual activity,
BRVO; branch retinal occlusion,
ETDRS; Early Treatment Diabetic Retinopathy Study,
IOP; intraocular pressure,
TA-LF; Triamcinolone acetomide loaded liposomes formulation,
†statistically significant differences from baseline values (P < 0.05),
‡no statistically significant differences from baseline values (P > 0.05).

### REFERENCES

1. Cugati S, Wang A J, Rochtchina E, Mitchell P. Ten-year incidence of retinal vein occlusion in an older population: the Blue Mountains Eye Study. Archives of ophthalmology 2006; 124:726-732.
2. Rogers S, McIntosh R L, Cheung N, et al. The prevalence of retinal vein occlusion: pooled data from population studies from the United States, Europe, Asia, and Australia. Ophthalmology 2010; 117:313-319. e311.
3. Rogers S L, McIntosh R L, Lim L, et al. Natural history of branch retinal vein occlusion: an evidence-based systematic review. Ophthalmology 2010; 117:1094-1101. e1095.
4. Hayreh S S, Rojas P, Podhajsky P, Montague P, Woolson R F. Ocular neovascularization with retinal vascular occlusion-III: incidence of ocular neovascularization with retinal vein occlusion. Ophthalmology 1983; 90:488-506.
5. Hayreh S S, Zimmerman M B. Fundus changes in central retinal vein occlusion. Retina (Philadelphia, Pa.) 2015; 35:29.
6. Kolar P. Risk factors for central and branch retinal vein occlusion: a meta-analysis of published clinical data. Journal of ophthalmology 2014; 2014.
7. O'Mahoney P R, Wong D T, Ray JG. Retinal vein occlusion and traditional risk factors for atherosclerosis. Archives of Ophthalmology 2008; 126:692-699.
8. Newman-Casey P A, Stem M, Talwar N, Musch D C, Besirli C G, Stein J D. Risk factors associated with developing branch retinal vein occlusion among enrollees in a United States managed care plan. Ophthalmology 2014; 121:1939-1948.
9. Kwon H J, Kang E C, Lee J, Han J, Song W K. Obstructive sleep apnea in patients with branch retinal vein occlusion: a preliminary study. Korean Journal of Ophthalmology 2016; 30:121-126.
10. Simmons N L, Joseph A, Baumal C R. Traumatic branch retinal vein occlusion with retinal neovascularization following inadvertent retrobulbar needle perforation. Ophthalmic Surgery, Lasers and Imaging Retina 2016; 47:191-193.
11. Chen S-N, Yang T-C, Lin J-T, Lian I-B. End stage renal disease as a potential risk factor for retinal vein occlusion. Medicine 2015; 94.
12. Szigeti A, Schneider M, Ecsedy M, Nagy Z Z, Récsán Z. Association between retinal vein occlusion, axial length and vitreous chamber depth measured by optical low coherence reflectometry. BMC ophthalmology 2015; 15:45.
13. Bertelmann T, Bertelmann I, Szurman P, et al. Vitreous body and retinal vein occlusion. Der Ophthalmologe: Zeitschrift der Deutschen Ophthalmologischen Gesellschaft 2014; 111:1178-1182.
14. Shih C-H, Ou S-Y, Shih C-J, Chen Y-T, Ou S-M, Lee Y-J. Bidirectional association between the risk of comorbidities and the diagnosis of retinal vein occlusion in an elderly population: a nationwide population-based study. International journal of cardiology 2015; 178:256-261.
15. Zhou J Q, Xu L, Wang S, et al. The 10-year incidence and risk factors of retinal vein occlusion: the Beijing eye study. Ophthalmology 2013; 120:803-808.
16. Arnarsson A, Stefansson E. Laser treatment and the mechanism of edema reduction in branch retinal vein occlusion. Investigative ophthalmology & visual science 2000; 41:877-879.
17. Saika S, Tanaka T, Miyamoto T, Ohnishi Y. Surgical posterior vitreous detachment combined with gas/air tamponade for treating macular edema associated with branch retinal vein occlusion: retinal tomography and visual outcome. Graefe's archive fir clinical and experimental ophthalmology 2001; 239:729-732.
18. Silva R M, de Abreu J F, Cunha-Vaz J. Blood-retina barrier in acute retinal branch vein occlusion. Graefe's archive for clinical and experimental ophthalmology 1995; 233:721-726.
19. Noma H, Funatsu H, Yamasaki M, et al. Aqueous humour levels of cytokines are correlated to vitreous levels and severity of macular oedema in branch retinal vein occlusion. Eye 2008; 22:42-48.
20. Noma H, Funatsu H, Mimura T, Eguchi S, Shimada K. Role of soluble vascular endothelial growth factor receptor-2 in macular oedema with central retinal vein occlusion. British journal of ophthalmology 2011; 95:788-792.
21. Noma H, Mimura T, Yasuda K, Shimura M. Cytokine kinetics after monthly intravitreal bevacizumab for retinal vein occlusion associated with macular oedema. Ophthalmic research 2016; 56:207-214.
22. Noma H, Mimura T, Shimada K. Role of inflammation in previously untreated macular edema with branch retinal vein occlusion. BMC ophthalmology 2014; 14:67.
23. Kim J H, Shin J P, Kim I T, Park D H. Aqueous angiopoietin-like 4 levels correlate with nonperfusion area and macular edema in branch retinal vein occlusion. Investigative ophthalmology & visual science 2016; 57:6-11.
24. Xin X, Rodrigues M, Umapathi M, et al. Hypoxic retinal Müller cells promote vascular permeability by HIF-1-dependent up-regulation of angiopoietin-like 4. Proceedings of the National Academy of Sciences 2013; 110:E3425-E3434.
25. Regnier S A, Larsen M, Bezlyak V, Allen F. Comparative efficacy and safety of approved treatments for macular oedema secondary to branch retinal vein occlusion: a network meta-analysis. BMJ open 2015; 5:e007527.
26. Li J, Paulus Y M, Shuai Y, Fang W, Liu Q, Yuan S. New developments in the classification, pathogenesis, risk factors, natural history, and treatment of branch retinal vein occlusion. Journal of ophthalmology 2017; 2017.
27. Tadayoni R, Waldstein S M, Boscia F, et al. Individualized Stabilization Criteria-Driven Ranibizumab versus Laser in Branch Retinal Vein Occlusion: Six-Month Results of BRIGHTER. Ophthalmology 2016; 123:1332-1344.
28. Clark W L, Boyer D S, Heier J S, et al. Intravitreal aflibercept for macular edema following branch retinal vein occlusion: 52-week results of the VIBRANT study. Ophthalmology 2016; 123:330-336.
29. Glanville J, Patterson J, McCool R, Ferreira A, Gairy K, Pearce I. Efficacy and safety of widely used treatments for macular oedema secondary to retinal vein occlusion: a systematic review. BMC ophthalmology 2014; 14:7.
30. Lyall D A, Tey A, Foot B, et al. Post-intravitreal anti-VEGF endophthalmitis in the United Kingdom: incidence, features, risk factors, and outcomes. Eye (Lond) 2012; 26:1517-1526.
31. Poku E, Rathbone J, Wong R, et al. The safety of intravitreal bevacizumab monotherapy in adult ophthalmic conditions: systematic review. BMJ Open 2014; 4:e005244.
32. Fung A E, Rosenfeld P J, Reichel E. The International Intravitreal Bevacizumab Safety Survey: using the internet to assess drug safety worldwide. Br J Ophthalmol 2006; 90:1344-1349.
33. Arikan G, Osman Saatci A, Hakan Oner F. Immediate intraocular pressure rise after intravitreal injection of ranibizumab and two doses of triamcinolone acetonide. Int J Ophthalmol 2011; 4:402-405.
34. Chan C K, Fan D S, Chan W M, Lai W W, Lee V Y, Lam D S. Ocular-hypertensive response and corneal endothelial changes after intravitreal triamcinolone injections in Chinese subjects: a 6-month follow-up study. Eye (Lond) 2005; 19:625-630.
35. Veritti D, Di Giulio A, Sarao V, Lanzetta P. Drug safety evaluation of intravitreal triamcinolone acetonide. Expert Opin Drug Saf 2012; 11:331-340.
36. Bisht R, Mandal A, Jaiswal J K, Rupenthal I D. Nanocarrier mediated retinal drug delivery: overcoming ocular barriers to treat posterior eye diseases. Wiley Interdiscip Rev Nanomed Nanobiotechnol 2018; 10.
37. Duncan R, Gaspar R. Nanomedicine(s) under the microscope. Mol Pharm 2011; 8:2101-2141.
38. Klibanov A L, Maruyama K, Torchilin V P, Huang L. Amphipathic polyethyleneglycols effectively prolong the circulation time of liposomes. FEBS Lett 1990; 268:235-237.
39. Lopez-Berestein G, Mehta R, Hopfer R, Mehta K, Hersh E M, Juliano R. Effects of sterols on the therapeutic efficacy of liposomal amphotericin B in murine candidiasis. Cancer Drug Deliv 1983; 1:37-42.
40. Oku N, Nojima S, Inoue K. Selective release of non-electrolytes from liposomes upon perturbation of bilayers by temperature change or polyene antibiotics. Biochim Biophys Acta 1980; 595:277-290.
41. Allen T M, Cullis P R. Drug delivery systems: entering the mainstream. Science 2004; 303:1818-1822.
42. van Rooijen N, van Nieuwmegen R. Liposomes in immunology: multilamellar phosphatidylcholine liposomes as a simple, biodegradable and harmless adjuvant without any immunogenic activity of its own. Immunol Commun 1980; 9:243-256.
43. Di Tommaso C, Bourges J L, Valamanesh F, et al. Novel micelle carriers for cyclosporin A topical ocular delivery: in vivo cornea penetration, ocular distribution and efficacy studies. Eur J Pharm Biopharm 2012; 81:257-264.
44. Hathout R M, Mansour S, Mortada N D, Guinedi A S. Liposomes as an ocular delivery system for acetazolamide: in vitro and in vivo studies. AAPS PharmSciTech 2007; 8:1.
45. Altamirano-Vallejo J C, Navarro-Partida J, Gonzalez-De la Rosa A, et al. Characterization and Pharmacokinetics of Triamcinolone Acetonide-Loaded Liposomes Topical Formulations for Vitreoretinal Drug Delivery. J Ocul Pharmacol Ther 2018; 34:416-425.
46. Gonzalez-De la Rosa A, Navarro-Partida J. Altamirano-Vallejo J C, et al. Novel Triamcinolone Acetonide-Loaded Liposomes Topical Formulation for the Treatment of Cystoid Macular Edema After Cataract Surgery: A Pilot Study. J Ocul Pharmacol Ther 2019.
47. Mansoor S, Kuppermann B D, Kenney M C. Intraocular sustained-release delivery systems for triamcinolone acetonide. Pharm Res 2009; 26:770-784.
48. Shubber S, Vllasaliu D, Rauch C, Jordan F, Ilium L, Stolnik S. Mechanism of mucosal permeability enhancement of CriticalSorb® (Solutol® HS15) investigated in vitro in cell cultures. Pharm Res 2015; 32:516-527.
49. Purslow C, Wolffsohn J S. Ocular surface temperature: a review. Eye Contact Lens 2005; 31:117-123.
50. Shabir G A. Validation of high-performance liquid chromatography methods for pharmaceutical analysis. Understanding the differences and similarities between validation requirements of the US Food and Drug Administration, the US Pharmacopeia and the International Conference on Harmonization. J Chromatogr A 2003; 987:57-66.

What is claimed is:

1. A method of treating macular thickening and or macular cysts occurring after a branch retinal vein occlusion event in a patient in need of treatment thereof comprising administering a therapeutically effective amount of a topical ophthalmic formulation comprising an active ingredient and a thermodynamically stable liposome

2. The method according to claim 1 wherein the active ingredient is selected from a steroid.

3. The method according to claim 1 wherein the thermodynamically stable liposome is selected from PEG-12 glyceryl dimyristate.

4. The method according to claims 1 to 3 wherein the treatment results in an improvement in a visual outcome selected from the group consisting of visual acuity and contrast sensitivity after said branch retinal vein occlusion event.

5. The method according to claim 2 wherein the steroid is selected from triamcinolone acetonide.

6. The method according to claim 5 wherein the formulation comprises a topically administrable acetonide-loaded liposomes ophthalmic formulation comprising:
a) Triamcinolone acetonide (TA) from 0.01 to 2.00% (w/v).
b) Polyethyleneglycol (PEG-12) glyceryl dimyristate from 5-15% (w/v)
c) Ethyl alcohol from 0.7 to 2.1% (v/v)
d) Polyethylene glycol (15)-hydroxystearate (Kolliphor HS 15) 2.5-7.5% (w/v)
e) Citric acid anhydrous from 0.04-0.16% (w/v)
f) Sodium citrate dehydrate from 0.23-0.69% (w/v)
g) Benzalkonium chloride from 0.001-0.015% (w/v).
h) Grade 2 purified water.

7. The method according to claim 6 wherein the pH of the formulation is between 5 and 6 and wherein the viscosity is about 60-80 cP and the osmolarity is about 300-350 mOsm/L.

8. The method according to claim 7 wherein the pH is about 5.8, the viscosity is about 70 cP and the osmolarity is about 334 mOsm/L.

9. The method according to claim 6 wherein the concentration of triamcinolone acetonide is about 1 to 3 mg/mL.

10. The method according to claim 9 wherein the concentration of triamcinolone acetonide is about 2 mg/mL.

11. The method according to claim 5 wherein the formulation is characterized by having an in vitro diffusion chamber result showing TA concentration (μg/mL) over time 2 hrs to 10 hrs as

| Time | |
|---|---|
| (hours) | TA Concentration (μg/mL) |
| 2 | 0.22 |
| 3 | 0.35 |
| 4 | 0.5 |
| 5 | 0.65 |
| 6 | 0.8 |
| 7 | 0.95 |
| 8 | 1.1 |

12. A method of treating a patient having macular edema secondary to branch retinal vein occlusion comprising topically administering a pharmaceutically effective amount of a triamcinolone acetonide-loaded liposomal formulation as primary therapy for said patient.

13. The method according to claim 12 wherein the liposome comprises PEG-12 glyceryl dimyristate.

14. The method according to claim 12 wherein the topical formulation is applied as a 0.2% solution (one drop) six times per day to said patients eye.

15. The method according to claim 12 wherein the patient showed significant improvement of BVCA and CFT without significant IOP modification.

16. The method according to claim 12 wherein the treated eyes showed BCVA improvement from 40+/−12.0 to 64+/−15.9 letters and CFT reduction from about 682+/−278 to 271+/−57 μm after 12 weeks of therapy.

17. A method of increasing the average size of a liposome along with increasing the number of particles with the greater average size comprising increasing the concentration of triamcinolone acetonide in the liposomal composition.

18. The method according to claim 17 wherein aqueous solutions of TA were prepared by adding ultrapure water (UPW) to obtain varying concentrations selected from 0.2, 0.4, 1.0 and 1.4% followed by emulsion preparation using said concentrations to form a loaded liposome of 0.2, 0.4, 1.0 and 1.4% (w/w)TA/LF.

19. The method according to claim 17 wherein the average particle size of a 0.2% loaded formulation comprises 147+/−6 nm with 0% of particles greater than 1,000 nm.

20. The method according to claim 17 wherein the average particle size of a 1.4% TA loaded liposome is about 1443+/−71 nm with greater than about 50% having a particle size greater than 1,000 nm.
